(19) United States
(12) Patent Application Publication
(10) Pub. No.: US 2024/0415994 A1
(43) Pub. Date: Dec. 19, 2024

(54) TRANSPORTABLE MICROBIAL CONTROL SYSTEM

(71) Applicant: SMARTWASH SOLUTIONS, LLC, Salinas, CA (US)

(72) Inventors: Christopher Michael MCGINNIS, Salinas, CA (US); James M. BRENNAN, Pleasanton, CA (US); Eric Child WILHELMSEN, Milpitas, CA (US); Greg ROLSKY, Carmel Valley, CA (US)

(21) Appl. No.: 18/739,452

(22) Filed: Jun. 11, 2024

Related U.S. Application Data
(60) Provisional application No. 63/508,576, filed on Jun. 16, 2023.

(51) Int. Cl. A61L 2/20; A61L 2/00

(52) U.S. Cl. CPC A61L 2/202; A61L 2/0047; A61L 2202/25

(57) ABSTRACT

Techniques and apparatus for microbial control of an interior of an enclosure in a processing facility are provided. An example apparatus generally includes an oxidant generator and an interlock. The oxidant generator is configured to generate an oxidizing agent in a gaseous state, removably fluidly couple to an interior of an enclosure of the processing facility, and distribute the oxidizing agent in the gaseous state to the interior of the enclosure when the oxidant generator is fluidly coupled to the interior of the enclosure. The interlock is operable to prevent generation of the oxidizing agent by the oxidant generator when the oxidant generator is not fluidly coupled to the interior of the enclosure.

### CROSS-REFERENCE TO RELATED APPLICATION(S)

The present application for patent claims the benefit of U.S. Provisional Patent Application Ser. No. 63/508,576, filed Jun. 16, 2023, which is hereby incorporated by reference herein in its entirety and for all applicable purposes.

### BACKGROUND

### Technical Field

Aspects of the present disclosure relate to microbial control and, more particularly, to microbial control within enclosures including one or more electronic components, especially enclosures used within a processing facility.

### Description of the Related Art

Electrical panels or enclosures including one or more components, such as electronic components, can harbor undesirable bacteria and other microorganisms (i.e., microbes). This is particularly harmful in food processing plants, medical manufacturing facilities, cosmetics manufacturing facilities, and the like. For example, although an electronic enclosure is typically not a primary food contact surface, the enclosure does have the potential to indirectly transfer microorganisms to food products within a food processing plant. The electronic enclosure may be fabricated to tolerate sanitation and exterior washing. However, even with these precautions, the electronic enclosure is still capable of creating an environment capable of generating microbial growth, in which case such microorganisms could be unintentionally transferred from the electronic enclosure to one or more primary food contact surfaces within the food processing plant. Similarly, while electronic enclosures are typically not in contact with products (e.g., pharmaceuticals or makeup) of a medical manufacturing facility or a cosmetics manufacturing facility, for example, the enclosures do have the potential to indirectly transfer microorganisms to products within a processing facility (e.g., a factory or laboratory) in these and other such industries.

In processing environments, especially in the food industry, margins are tight, and there is a desire to fully utilize equipment. This is especially true for safety equipment. It is therefore desirable to develop methods and apparatus to improve utilization of safety equipment.

### SUMMARY

The devices, apparatuses, systems, and methods of this disclosure each have several aspects, no single one of which is solely responsible for its desirable attributes. Without limiting the scope of this disclosure as expressed by the claims which follow, some features will now be discussed briefly. After considering this discussion, and particularly after reading the section entitled “Detailed Description” one will understand how the features of this disclosure provide advantages that include increased microbial lethality within an enclosure housing one or more electronic components.

Aspects of the present disclosure generally relate to microbial control within enclosures including one or more electronic components, especially enclosures used within a processing facility, such as a food processing facility, a manufacturing facility for medicines, or a cosmetics manufacturing facility.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, and an oxidant generator configured to generate an oxidizing agent in a gaseous state and distribute the oxidizing agent in the gaseous state within the interior of the enclosure.

Certain aspects of the present disclosure provide a microbial control system. The system includes an enclosure with a door movable with respect to the enclosure between an open position to enable access to an interior of the enclosure and a closed position to prevent access to the interior of the enclosure, an electronic component positioned within the interior of the enclosure, a switch operably coupled to the door to determine if the door is in the open position or the closed position with respect to the enclosure, and an ultraviolet (UV) light source positioned within the interior of the enclosure, configured to generate ozone in a gaseous state within the interior of the enclosure, and operably coupled to the switch such that the UV light source is configured to only operate when the door is in the closed position.

Certain aspects of the present disclosure provide a system for microbial control within an enclosure including an electronic component. The system includes an oxidant generator housing configured to be in fluid communication with an interior of an enclosure, an oxidant generator configured to generate an oxidizing agent in a gaseous state within an interior of the oxidant generator housing, and a pressure source in fluid communication with the interior of the oxidant generator housing and configured to provide a positive pressure in the interior of the oxidant generator housing and distribute the oxidizing agent in the gaseous state from the interior of the oxidant generator housing to the interior of the enclosure.

Certain aspects of the present disclosure provide a kit for microbial control within an enclosure. The kit includes a switch configured to operably couple to an access cover of the enclosure such that the switch is configured to be in a first position when the access cover is in an open position with respect to the enclosure and the switch is in a second position when the access cover is in a closed position with respect to the enclosure, and an oxidant generator configured to be positioned within an interior of the enclosure, generate an oxidizing agent in a gaseous state within the interior of the enclosure, and be operably coupled to the switch, wherein the switch is configured to at least one of: prevent operation of the oxidant generator when the switch is in the first position; or cause the oxidant generator to operate when the switch is in the second position.

Certain aspects of the present disclosure provide a method for controlling microbes. The method generally includes receiving a signal indicative of an access cover of an enclosure being closed to block access to an interior of the enclosure and controlling an oxidant generator, based on reception of the signal, to introduce an oxidizing agent into the interior of the enclosure.

Certain aspects of the present disclosure provide an apparatus for microbial control in a processing facility. The apparatus generally includes an oxidant generator and an interlock. The oxidant generator is configured to generate an oxidizing agent in a gaseous state, removably fluidly couple to an interior of an enclosure of the processing facility, and distribute the oxidizing agent in the gaseous state to the interior of the enclosure when the oxidant generator is fluidly coupled to the interior of the enclosure. The interlock is operable to prevent generation of the oxidizing agent by the oxidant generator when the oxidant generator is not fluidly coupled to the interior of the enclosure.

Certain aspects of the present disclosure provide an apparatus for microbial control in a processing facility comprising an enclosure having an access cover configured to be selectively opened to control access to an interior of the enclosure. The apparatus generally includes an oxidant generator and an interlock. The oxidant generator is configured to generate an oxidizing agent in a gaseous state, removably fluidly couple to the interior of the enclosure of the processing facility, and distribute the oxidizing agent in the gaseous state to the interior of the enclosure when the oxidant generator is fluidly coupled to the interior of the enclosure. The interlock is operable to prevent the access cover of the enclosure from being opened when the oxidant generator is fluidly coupled to the interior of the enclosure.

Aspects of the present disclosure generally include methods, apparatus, and systems, as substantially described herein with reference to and as illustrated by the accompanying drawings. Numerous other aspects are provided.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective aspects.

FIG. 1 is a schematic perspective view of an example microbial control system, in accordance with certain aspects of the present disclosure.

FIG. 2 is a schematic cutaway view of the microbial control system shown in FIG. 1.

FIG. 3 is a block diagram of an example microbial control system with a controller and sources of various signals, in accordance with certain aspects of the present disclosure.

FIG. 4 is a block diagram of an example microbial control system with one or more power sources, in accordance with certain aspects of the present disclosure.

FIG. 5 is a block diagram of an example microbial control system with a portion thereof positioned outside of an enclosure, in accordance with certain aspects of the present disclosure.

FIGS. 6A-6D illustrate example oxidant generators used to generate ozone, in accordance with certain aspects of the present disclosure.

FIGS. 7A-7C illustrate example oxidant generators used to generate chlorine dioxide, in accordance with certain aspects of the present disclosure.

FIG. 8 is a flow diagram illustrating example operations for controlling microbes, in accordance with certain aspects of the present disclosure.

FIG. 9 is a block diagram of an example portable microbial control device with an included interlock, the device being positioned outside of an enclosure, in accordance with certain aspects of the present disclosure.

FIG. 10 is a block diagram of an example portable microbial control device with an included locking mechanism, the device being positioned outside of an enclosure, in accordance with certain aspects of the present disclosure.

FIG. 11 is a block diagram of an example portable microbial control device with an included interlock and a locking mechanism, the device being positioned outside of an enclosure, in accordance with certain aspects of the present disclosure.

FIGS. 12A and 12B are perspective schematic views of an example portable microbial control device in different positions with respect to an enclosure, in accordance with certain aspects of the present disclosure.

To facilitate understanding, identical reference numerals have been used, where possible, to designate identical elements that are common to the figures. It is contemplated that elements described in one aspect may be beneficially utilized on other aspects without specific recitation.

### DETAILED DESCRIPTION

Certain aspects of the present disclosure provide apparatus, systems, and methods for microbial control within an enclosure in a processing facility, the enclosure housing one or more electronic components. One example system for providing microbial control to an enclosure generally includes the enclosure having an access cover configured to be selectively opened to enable access to an interior of the enclosure; an electronic component at least partially disposed in the interior of the enclosure, wherein the electronic component is operable to interact with the processing facility; and an oxidant generator configured to generate an oxidizing agent in a gaseous state and distribute the oxidizing agent in the gaseous state within the interior of the enclosure.

In aspects of the present disclosure, a device for microbial control of more than one enclosure may include certain portions that are portable while microbial control of the enclosure is maintained to improve user safety. Certain aspects of the present disclosure may reduce costs of processing facilities by permitting reuse or multiple uses of at least a portion of the device for microbial control. Methods and apparatus for improving user safety while providing microbial control are discussed below.

Certain aspects of the present disclosure provide various strategies for allowing a microbial control process for single enclosures to be used with multiple enclosures without increasing the exposure hazard to users. In one strategy, the device for microbial control can include a locking mechanism (e.g., an interlock) configured to lock an access cover of an enclosure while the device for microbial control is attached to the enclosure. For example, if the device is given interior access to the enclosure from outside and includes a mechanism for ensuring the enclosure cannot be opened while the device is operational, safety is assured. Having a connection for electrical power in the interior of the enclosure may improve portability of the device, but is optional. In another strategy, the device can be removably attached to the previously described interlocks of the enclosures. This attachment to an interlock of an enclosure may be optionally facilitated with various keyed electrical connections.

The following description provides examples, and is not limiting of the scope, applicability, or examples set forth in the claims. Changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method which is practiced using other structure, functionality, or structure and functionality in addition to or other than the various aspects of the disclosure set forth herein. It should be understood that any aspect of the disclosure described herein may be embodied by one or more elements of a claim. The word “exemplary” is used herein to mean “serving as an example, instance, or illustration.” Any aspect described herein as “exemplary” is not necessarily to be construed as preferred or advantageous over other aspects.

Certain aspects of the present disclosure provide features of microbial control systems that enable a device for microbial control of an enclosure to be portable.

Some features are common between inside-the-enclosure microbial control devices and outside-of-the-enclosure microbial control devices, whether portable or otherwise, but other features will differ. To implement features applying to outside-of-the-enclosure microbial control devices, modifications may be made to the enclosures to be treated by the microbial control devices.

In aspects of the present disclosure, an access port on the enclosure may be rescalable to maintain the integrity of the enclosure for sanitation when the portable microbial control device is not attached. There are many approaches to making a resealable access port on the enclosure. One exemplary approach to making the access port of an enclosure resealable may be based on various sanitary quick release couplings. Another exemplary approach to making the access port of an enclosure rescalable may include use of a threaded collar to squeeze an open metal cone into an aperture on a housing of the enclosure for a gastight gasket-less metal-on-metal seal. Such a seal may be similar to fittings used for drain plumbing.

According to aspects of the present disclosure, a power connection to provide electrical power to a microbial control device from an enclosure also may not violate the integrity of the enclosure. An external power source may meet this design specification, but providing an additional power outlet near each enclosure may not be desirable. There are a number of known electrical connections that are designed to work in process facilities. In some aspects of the present disclosure, the device includes a collared fitting, where an outside nut tightens down and both compresses the seal and causes the electrical connection to be completed. The case of use of a microbial control device may be improved if the act of attaching the microbial control device to the enclosure also attaches the power to the microbial control device. Such an electrical connection may be within a pipe or tube that fluidly couples an oxidant generator to the interior of the enclosure, or may run parallel to such a tube or pipe.

In aspects of the present disclosure, an external portion of a microbial control device may benefit from including indicator lights or other indicators of the status of the microbial control device and/or status of a process or treatment in progress. For example, a microbial control device may report (e.g., via indicator lights) when a process or treatment is in progress. It may additionally or alternatively be desirable for the microbial control device to report that the oxidant generator is idle, but that additional time is recommended to allow the oxidant to dissipate before removing the microbial control device and/or accessing the interior of the treated enclosure. It may additionally or alternatively be desirable for the microbial control device to report or indicate an all-clear signal when it is safe to move the microbial control device and/or access the interior of the treated enclosure. The three statuses described above (or a subset thereof) could be indicated, for example, with a set of light-emitting diodes (LEDs) of the same or different colors. Alternatively, these three statuses (or a subset thereof) could be indicated by one light that changes color and/or changes light emission patterns, according to the indicated status. For example, the light may change from a solid light to a blinking light to indicate corresponding changes in status.

Certain aspects of the present disclosure include logic and/or an interlock that may enhance user safety. The logic and/or interlock may prevent the oxidant generator from generating oxidant when the microbial control device is not attached to an appropriate enclosure. For example, an ultraviolet (UV) lamp used for generating ozone may be prevented from lighting when the microbial control device is not attached to an appropriate enclosure. The logic may also time the treatment in an effort to ensure that a treatment is sufficient. This logic may be similar to logic included with a non-portable microbial control device in many respects.

In some aspects of the present disclosure, a means of preventing the enclosure from being opened while a treatment by a microbial control device is in progress is provided. For example, a microbial control device may block access to a handle of an access panel of an enclosure when the microbial control device is coupled to and/or treating an enclosure. In another example, a microbial control device includes a latching mechanism that prevents an access cover of an enclosure from being opened when the microbial control device is coupled to and/or treating an enclosure. In yet another example, a microbial control device may include a physical bar that obstructs opening an access cover of an enclosure when the microbial control device is coupled to and/or treating an enclosure. In yet another example, a microbial control device may include one or more magnets that may ensure an enclosure does not open when the microbial control device is coupled to and/or treating the enclosure. These and/or other means of preventing the enclosure from being opened may be combined in some aspects of the present disclosure.

According to certain aspects of the present disclosure, a portable internal system (e.g., a portable microbial control device configured to operate within an enclosure) might include and benefit from many of the above-described features, but may use some different approaches.

In some aspects of the present disclosure, a portable microbial control device configured to operate within an enclosure may utilize an external locking system (e.g., similar to locking systems used for fixed microbial control systems) to ensure the enclosure remains closed when the microbial control device is coupled to and/or treating the enclosure.

In some aspects of the present disclosure, a portable microbial control device configured to operate within an enclosure may utilize a keyed plug as a connection to a power source.

According to certain aspects of the present disclosure, it may be desirable to report, outside of an enclosure, a device status of a portable microbial control device configured to operate within the enclosure. For example, the device status may indicate that a process or treatment is in progress, that the device is idle (but more waiting time is recommended), or that the interior of the enclosure may be accessed and/or the device may be removed (e.g., with an all-clear signal).

In both portable microbial control devices configured to operate within an enclosure (also referred to herein as “internal designs”) and portable microbial control devices configured to operate while outside an enclosure (also referred to herein as “external designs”), it may be desirable to include as many components and/or features as possible in the portable part relative to those components and/or features added to the various enclosures. Thus, it may be desirable for portable microbial control devices to include the oxidant generator (e.g., a UV lamp) and power supply in certain aspects of the present disclosure. It may also be desirable for portable microbial control devices to include logic (e.g., a controller, a timer, a sensor interface, and/or an interlock interface). It may also be desirable for portable microbial control devices to include as much of the safety and interlock hardware as practical. It also may be desirable for portable microbial control devices to include mechanical means to block an access cover of an enclosure from opening.

Electronic component enclosures are increasing in quantity and quality (e.g., sophistication) as electronic components increase in use in modern society. For example, as the Internet of things (IoT) continues to grow, along with the level and sophistication of automation, so will the number of electronic component enclosures. Controlling the growth and exposure to microorganisms with respect to these electronic component enclosures becomes increasingly important, particularly in industries for food processing, medical applications, and for manufacturing products such as drugs, dietary supplements, medical materials, or consumables.

Further, factors facilitating microbial growth are enhanced with potential variable temperatures and other environment changes for the electronic component enclosures. For example, cold temperatures used within a food processing environment may cause a box to “breathe” as temperatures vary with air and gas being drawn into and expelled from the electronic component enclosure. This movement of gas into and out of the electronic component enclosure increases the ability for microorganisms to transfer into and out of the electronic component enclosure. Adding moisture and/or using the electronic component enclosures within a wet environment also adds another risk factor for facilitating microbial growth. For example, the food processing environment often incorporates both cold and wet environments, and electronic component enclosures are often opened periodically during use, introducing even further risk.

Thus, aspects of the present disclosure generally relate to microbial control within a system including an enclosure. The enclosure includes an interior with one or more electronic components positioned within the interior of the enclosure. The electronic component(s) may include a distribution board (a panel board, breaker panel, or electric panel), a semiconductor component, an electronic circuit, an integrated circuit (IC), a controller or processor, a power converter (e.g., a voltage regulator), and/or one or more other types of electronic components. The enclosure includes an access feature (e.g., a door or access panel) that is movable (or removable) to enable access to the interior of the enclosure, such as for accessing and checking or interacting with the electronic component(s). Further, an oxidant generator is in use with the enclosure, such as to generate an oxidizing agent and distribute the oxidizing agent within the interior of the enclosure. The oxidant generator may be used to generate the oxidizing agent in a gaseous state for distribution within the interior of the enclosure. The oxidant generator may be positioned within the interior of the enclosure. Alternatively, the oxidant generator may be positioned external to the interior of the enclosure, but may be in fluid communication with the interior of the enclosure, such as to have the oxidizing agent in the gaseous form routed to the interior of the enclosure from a separate housing or location. The oxidizing agent is able to interact with microorganisms to oxidize, control, and kill the microorganisms. Thus, microbes, such as listeria and/or mold, may be controlled and prevented from growth by distributing the oxidizing agent within the interior of the enclosure.

### Examples of Microbial Control for an Enclosure

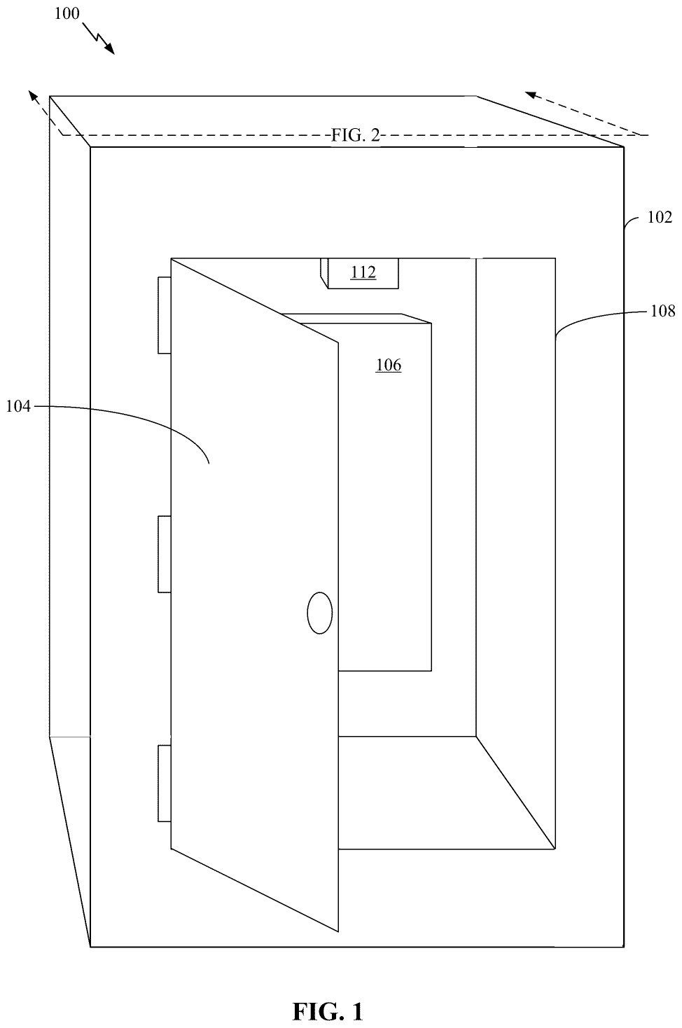

Referring now to FIG. 1, a schematic view of a microbial control system 100 in accordance with one or more aspects of the present disclosure is shown. The system 100 may be used within any system or environment (e.g., a process facility) where it is desired to control microorganisms within an enclosure 102. Accordingly, the system 100 may be used within a food processing system, a medical application system, or another system, such as for processing products that include drugs, dietary supplements, medical materials, or consumables. The system 100 includes the enclosure 102 with an access cover 104 (e.g., a door or sliding access panel) movable (or removable) to enable access to an interior of the enclosure 102. An electronic component 106 is at least partially positioned within the interior of the enclosure 102 such that the electronic component 106 is at least partially housed within and protected by the enclosure 102. Although only one electronic component 106 is shown in FIG. 1, the reader is to understand that there may be more than one electronic component disposed within the interior of the enclosure 102. An example of an electronic component 106 may include a controller of an Automated SmartWash Analytical Platform (ASAP)™, available from SmartWash Solutions, LLC of Salinas, California, and described in U.S. Patent Application Publication No. 2018/0093901 to Brennan et al., filed on Oct. 3, 2017 and entitled “System for Controlling Water Used for Industrial Food Processing,” which is incorporated by reference herein in its entirety. The access cover 104 enables access to the electronic component 106 within the interior of the enclosure 102, such as when interacting with the electronic component 106, for example, for maintaining or replacing the electronic component 106.

The access cover 104 is movable (or removable) between an open position and a closed position with respect to the enclosure 102. The open position for the access cover 104 is shown in FIG. 1. In the open position, the access cover 104 enables access to the interior of the enclosure 102 through an opening 108, such as for interacting with the electronic component 106. In the closed position, the access cover 104 may be secured to the enclosure 102 to enclose and seal the interior of the enclosure 102 and prevent access to the interior of the enclosure 102.

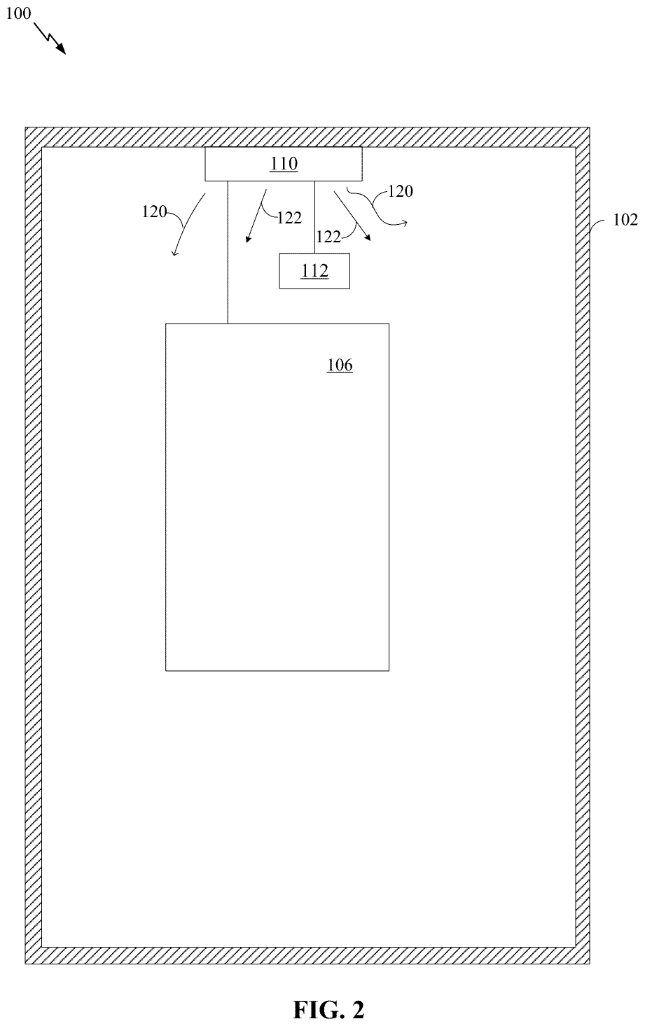

The system 100 may further include a switch 112 that is operably coupled to the access cover 104 such that the switch is in a first position when the access cover 104 is in the open position with respect to the opening 108 of the enclosure 102 and the switch is in a second position when the access cover 104 is in a closed position with respect to the opening 108. As discussed in more detail below with reference to FIG. 2, when the switch 112 is in the first position, an oxidant generator in the enclosure 102 is prevented from operating, and when the switch 112 is in the second position, the oxidant generator in the enclosure 102 may operate. An example of the switch 112 may include a switch relay, such as a double-pole switch relay. The switch 112 may be positioned adjacent the access cover 104 and/or the opening 108 to measure a position of the access cover 104 with respect to the opening 108 and/or the enclosure 102. In another aspect, as the access cover 104 may be rotatable with respect to the enclosure 102 to move between the open position and the closed position (e.g., in the case of a door on hinges), the switch 112 may be able to measure the amount of rotation between the access cover 104 and the enclosure 102. Further, in another aspect, a latch or lock may be used with the access cover 104 to secure the access cover 104 in the closed position with respect to the opening 108 of the enclosure 102. In such an aspect, the switch 112 may be operably coupled to the latch or lock such that the switch is in the first position when the access cover 104 is in the closed position, but not secured in the closed position with the latch or lock. That is, the switch 112 may be in the first position that prevents an oxidant generator in the enclosure 102 from operating, when the access cover 104 is closed, but not secured with the latch. When both the access cover 104 is in the closed position and the latch or lock is in the secured position, the switch 112 may be in the second position, allowing or causing the oxidant generator to operate. Furthermore, in another aspect, the switch 112 (or a sensor coupled thereto) may be able to detect a presence of external light, such as natural light, being received into the interior of the enclosure 102. In such an aspect, the switch 112 may be in the first position if external light is received within the enclosure 102.

FIG. 2 is a schematic cutaway view of the microbial control system 100, in accordance with aspects of the present disclosure. An oxidant generator 110 is included with the microbial control system 100. In FIG. 2, the oxidant generator 110 is shown positioned within the interior of the enclosure 102 to distribute the oxidizing agent within the interior of the enclosure 102. The oxidant generator 110 may be used to generate one or more oxidizing agents 120 in a gaseous state and then distribute the one or more oxidizing agents 120 within the interior of the enclosure 102. The one or more oxidizing agents 120 from the oxidant generator 110 may be used to oxidize existing microorganisms included within the enclosure 102, and/or may be used to prevent growth of microorganisms within the enclosure 102, thereby preventing the enclosure 102 with the electronic component 106 from being a potential microbial source that may contaminate a larger system or facility that uses the enclosure 102 with the electronic component 106. For certain aspects, the oxidant generator 110 may additionally or alternatively generate electromagnetic radiation (e.g., ultraviolet (UV) light) 122 that may kill or inactivate microorganisms in the enclosure 102.

Generating the oxidizing agent 120 in a gaseous state, as performed by the oxidant generator 110, may enable the antimicrobial properties of the oxidizing agent to be distributed within the enclosure 102 and may be suitable for the safety of a system or facility incorporating the electronic component 106, such as a food processing system or other system where microbial contamination should be avoided (e.g., a system for processing drugs, medical materials, or cosmetics). In one aspect, the oxidizing agent 120 includes ozone such that the oxidant generator 110 is an ozone generator to generate ozone. In another aspect, the oxidizing agent 120 includes chlorine dioxide such that the oxidant generator 110 is a chlorine dioxide generator to generate chlorine dioxide.

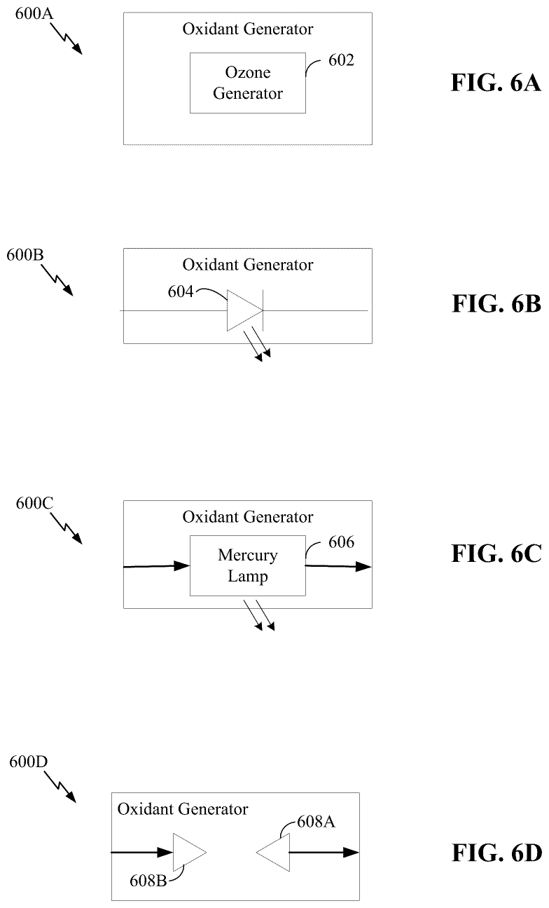

In one aspect, ozone may be generated from infusing energy with oxygen in the air. Further, after ozone dissipates, the ozone may leave substantially no residue behind. FIGS. 6A-6D show various examples of oxidant generators 600A-600D that may be used to generate ozone and may be considered examples of the oxidant generator 110 illustrated in FIG. 2. In FIG. 6A, as discussed, the oxidant generator 600A may include an ozone generator 602 used to generate ozone. In one or more aspects, ozone may be generated (e.g., from ultraviolet (UV) light or an electrical discharge) at a low but lethal level for microbial control. Thus, the ozone generator 602 may include a UV light source and/or an electrical discharge source. FIG. 6B shows an example of a UV light source for an oxidant generator 600B that includes at least one light-emitting diode (LED) 604 for generating UV light. FIG. 6C shows an example of a UV light source for an oxidant generator 600C that includes a mercury lamp 606 for generating UV light. Other examples of UV light sources may also be used for an ozone generator, such as other types of lamps or bulbs, without departing from the scope of the present disclosure.

As discussed, the UV light source generates electromagnetic radiation that may interact with oxygen in the air to create ozone. Further, the UV light source itself, in addition to the ozone created by the UV light source, may have antimicrobial properties to kill microorganisms. For example, though the antimicrobial properties of the UV light are limited to areas in a line of sight from the UV light source and thus those microorganisms that are shaded from the UV light source may remain unaffected, ozone generated by the UV light source may be able to kill and destroy microorganisms that are shaded from the UV light source.

A UV light source may produce UV light having a wavelength (λ) between about 10 nm and 400 nm (e.g., about 240 nm). In one or more aspects of the present disclosure and based upon several factors, such as the amount of ozone being generated and/or the size of the interior of the enclosure 102, the UV light source may use between about five watts (W) to about twenty-five watts of electrical power (e.g., about 6 W). A UV light source of this power level may be able to sanitize and control microorganisms in a ten cubic foot (10 ft³) (=0.28 cubic meters (m³)) enclosure in less than about one hour (e.g., about 52 minutes). If the treatment is continuous from the UV light source, or any oxidant generator 110 in general, microorganisms, and listeria specifically, may not be able to form colonies in the enclosure 102. If the enclosure 102 is rarely opened, a timer, discussed in more detail below, may be used to reduce power consumption and/or extend the lifetime of the oxidant generator 110. For example, a UV light source may have an operating life of about 10,000 hours, so a timer may extend the useful life of the UV light source by causing the UV light source to be on often enough to prevent microorganisms from forming colonies in the enclosure while preventing the UV light source from continuously operating.

An example is shown in FIG. 6D of an oxidant generator 600D including an electrical discharge source. In this aspect, the electrical discharge source may include one or more electrodes, such as a pair of electrodes 608. An electric spark may be generated in the gap between the pair of the electrodes 608, in which the energy of the electrical discharge may interact with oxygen in the air to create ozone. This type of arrangement with a pair of electrodes 608 separated by a gap may be referred to as a “spark gap.”

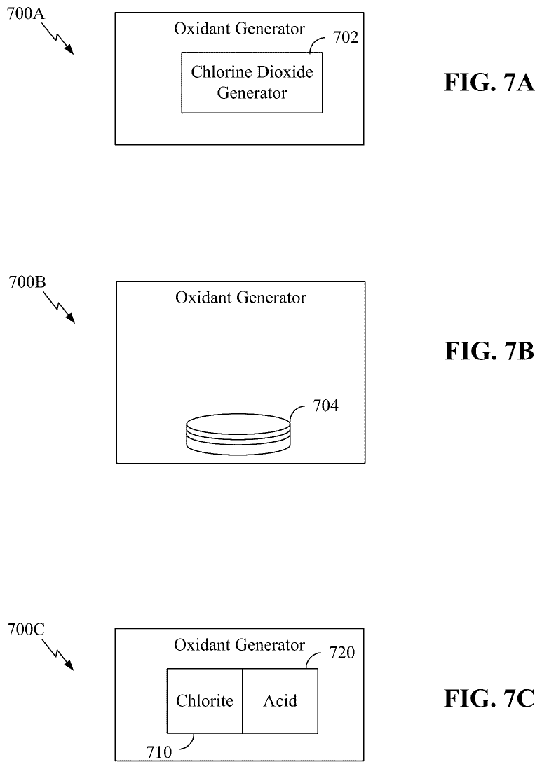

The oxidant generator 110 may additionally or alternatively include a chlorine dioxide generator to generate chlorine dioxide. FIGS. 7A-7C show various examples of oxidant generators 700A-700C that may be used to generate chlorine dioxide. In FIG. 7A, the oxidant generator 700A may include a chlorine dioxide generator 702 used to generate chlorine dioxide. In one or more aspects, the chlorine dioxide generator 702 may be configured to cause multiple chemicals to react with each other and generate chlorine dioxide.

FIG. 7B shows an example of a chemical component used for, or as a part of, an oxidant generator 700B in the form of a tablet 704. The tablet 704 may include or be formed from a chlorite, such as sodium chlorite or potassium chlorite. The tablet 704 may interact with another chemical, such as acid or water, to generate chlorine dioxide. The tablet 704 may be released from an inert storage (e.g., from a sealed bag) to begin reacting with another chemical manually, such as by an operator removing the tablet 704 from a sealed package and positioning the tablet 704 upon a holder or tray within an oxidant generator. The tablet 704 may alternatively be released automatically, such as by having a motor open a cover of a sealed chamber or compartment containing the tablet 704 to release the tablet 704 and cause the tablet 704 to be exposed to or interact with acid or water. The acid or water may also be released manually or automatically, similar to the tablet 704. Further, the tablet 704 may be able to interact with moisture in the air to generate chlorine dioxide, as opposed to having to introduce the water separately.

FIG. 7C shows a schematic of an exemplary oxidant generator 700C including a chlorite 710 used to interact with an acid 720 to generate chlorine dioxide. The chlorite 710 may be in the form of the tablet 704 (shown in FIG. 7B). The acid 720 may be hydrochloric acid or sulfuric acid, for example.

Returning to FIG. 2, the oxidant generator 110 may be operably coupled to the switch 112 such that the operation of the oxidant generator 110 may be controlled based upon the state of the switch 112. For example, in some aspects, the switch 112 may be in a first position when the access cover 104 (see FIG. 1) is in the closed position and/or when the access cover 104 is secured in the closed position, and the switch 112 may be in a second position when the access cover 104 is in the open position or when the access cover 104 is not secured (e.g., not latched) in the closed position. In the example, the switch 112 or the state thereof may prevent the oxidant generator 110 from operating when the switch is in the second position (e.g., when the access cover 104 is in the open position or the access cover 104 is not secured in the closed position). In such an aspect, the switch 112 may be operably coupled to the access cover 104, to the opening 108, or to a securing mechanism (e.g., a latch or lock) coupled to the access cover 104 or the enclosure 102. This control of the operation of the oxidant generator 110 may increase the effectiveness of the microbial control for the oxidant generator 110 within the enclosure 102, and may provide a safety barrier for those that interact with the enclosure 102.

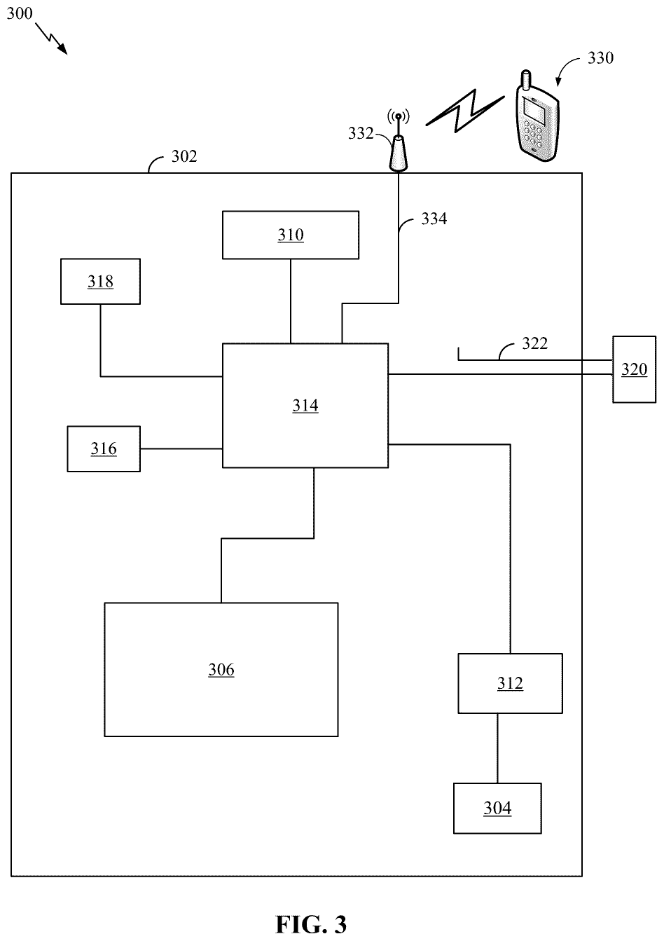

Referring now to FIG. 3, a block diagram of an example microbial control system 300 in accordance with one or more aspects of the present disclosure is shown. The system 300 includes an enclosure 302 with an access cover 304 movable (or altogether removable) to enable access to an interior of the enclosure 302 and at least one electronic component 306 positioned within the interior of the enclosure 302. An example of an electronic component 306 may include a controller of an Automated SmartWash Analytical Platform (ASAP)™, available from SmartWash Solutions, LLC of Salinas, California, and described in U.S. Patent Application Publication No. 2018/0093901 to Brennan et al., filed on Oct. 3, 2017 and entitled “System for Controlling Water Used for Industrial Food Processing.” An oxidant generator 310 is also positioned within the interior of the enclosure 302 to distribute the oxidizing agent within the interior of the enclosure 302. A switch 312 is also included with the system 300 by being operably coupled to the oxidant generator 310.

Further, the system 300 includes a controller 314 operably coupled to the oxidant generator 310 with the controller 314 including or being operably coupled to one or more other components. As shown, the controller 314, which may be a programmable logic controller (PLC), for example, is operably coupled to the electronic component 306 and the switch 312, and may also be operably coupled to (or include) a timer 316, a sensor 318, an antenna 332, and/or a user interface 330. The user interface 330 may wirelessly communicate with the controller 314 via an antenna 332 that is coupled to the controller via a wire 334 and a transceiver (not shown), or alternatively, the user interface 330 may be connected (or otherwise coupled) to the controller via a wire (not shown). For example, the timer 316, which may be a timer relay, may generate a timer signal (e.g., a first signal) to control the operation of the oxidant generator 310. Additionally or alternatively, the sensor 318 may generate a sensor signal (e.g., a second signal) to control the operation of the oxidant generator 310, and/or the user interface 330 may generate a user input signal (e.g., a third signal) to control the operation of the oxidant generator 310 via the antenna 332 and wire 334. The controller 314 may be operably coupled between the electronic component 306, the oxidant generator 310, the switch 312, the timer 316, the sensor 318, the antenna 332, and/or the user interface 330 and may be programmed to control the oxidant generator 310 based on a switch signal from the switch 312, the timer signal from the timer 316, the sensor signal from the sensor 318, and/or the user input signal from the user interface 330 or antenna 332. As the controller 314 is operably coupled to the electronic component 306, the oxidant generator 310, the switch 312, the timer 316, the sensor 318, the antenna 332, and/or the user interface 330, the controller 314 may be wired and/or wirelessly connected with (or otherwise coupled to) each of these components to facilitate communication and control therebetween.

As shown, the sensor 318 may be positioned within the enclosure 302 and may be used to measure the oxidizing agent within the interior of the enclosure 302. The sensor 318 may be used to measure the presence of the oxidizing agent within the enclosure 302 and/or the amount or concentration of the oxidizing agent within the enclosure 302. The sensor 318 (e.g., in conjunction with the controller 314) may be used to control the operation of the oxidant generator 310 and/or may be able to determine if the oxidant generator 310 is working properly. For example, the oxidizing agent may be generated and distributed by the oxidant generator 310 within the interior of the enclosure 302 at a predetermined rate or at a predetermined concentration. The sensor 318 may be used to verify or control the oxidant generator 310 based upon a comparison of the measured rate or concentration of the oxidizing agent within the interior of the enclosure 302 and the predetermined rate, the predetermined concentration, or a threshold (e.g., a minimum or a maximum) concentration.

Further, the oxidizing agent may be a dangerous agent, such that for those (e.g., facility personnel) working in proximity to the oxidizing agent, the amount or level of oxidizing agent is moderated or even regulated in the work place by the Occupational Safety and Health Administration (OSHA) or a similar regulatory agency. The oxidizing agent may soften plastic and/or insulation by breaking down polymers, and thus may also be destructive for the enclosure and/or the electronic component(s) within the enclosure. Thus, it is desirable that the oxidant generator provide a quantity of oxidizing agent sufficient to sanitize and control the microorganisms within the enclosure 302, but not so much that the oxidizing agent damages the enclosure 302 and/or related equipment, possibly resulting in a premature failure. Thus, the sensor 318 may be used to facilitate monitoring of the oxidizing agent produced by the oxidant generator 310.

Referring still to FIG. 3, a pressure source 320, such as a pump, may be operably coupled with the interior of the enclosure 302 to provide a positive pressure in the interior of the enclosure 302 for certain aspects. The pressure source may generate a positive pressure (e.g., pumping air, nitrogen, or another gas into the enclosure 302) by providing pressure into the interior of the enclosure 302 to generate a higher pressure within the interior of the enclosure 302 than a pressure exterior to the enclosure 302. The pressure source 320 may be positioned within, or partially within, the enclosure 302 to provide the positive pressure within the enclosure 302. Alternatively, the pressure source 320 may be positioned exterior to the enclosure 302 with the pressure source in fluid communication with the interior of the enclosure 302. The pressure source 320 may be in fluid communication with the interior of the enclosure 302 by having the gas routed through a flow line 322 (e.g., a pipe or tubing), as shown, to provide the positive pressure from the pressure source to the interior of the enclosure 302. Further, the pressure source 320 may be operably coupled to the controller 314, as shown, such that the controller 314 is programmed to control the operation of the pressure source. For example, the controller 314 may be used to control the operation of the pressure source 320 based upon the operation of the oxidant generator 310 such that the pressure source 320 and the oxidant generator 310 operate concurrently or overlap in their operating times.

As described above, the pressure source 320 may be used to create a positive pressure environment within the interior of the enclosure 302. A positive pressure environment may facilitate microbial control within the enclosure 302, such as by preventing air or another gas from entering the interior of the enclosure 302, due to the pressure difference between the interior and the exterior of the enclosure 302 causing air and other fluids to flow out of the enclosure 302 and not into the enclosure 302. In one aspect, the pressure source 320 may create a positive pressure of about four inches of water pressure (about 1 kilopascal). Further, depending on the size of the enclosure 302, the pressure source may be able to pump air or gas at about one to two cubic feet per hour (about 0.028 to 0.057 m³ per hour) into the interior of the enclosure 302. Furthermore, the pressure source 320 may provide gas pressure through the oxidant generator 310 to facilitate distribution of the oxidizing agent within the interior of the enclosure 302. For example, gas pressure from the pressure source 320 may be provided between the pair of electrodes 608 (shown in FIG. 6D) to distribute the ozone from the pair of electrodes 608 within the interior of the enclosure 302.

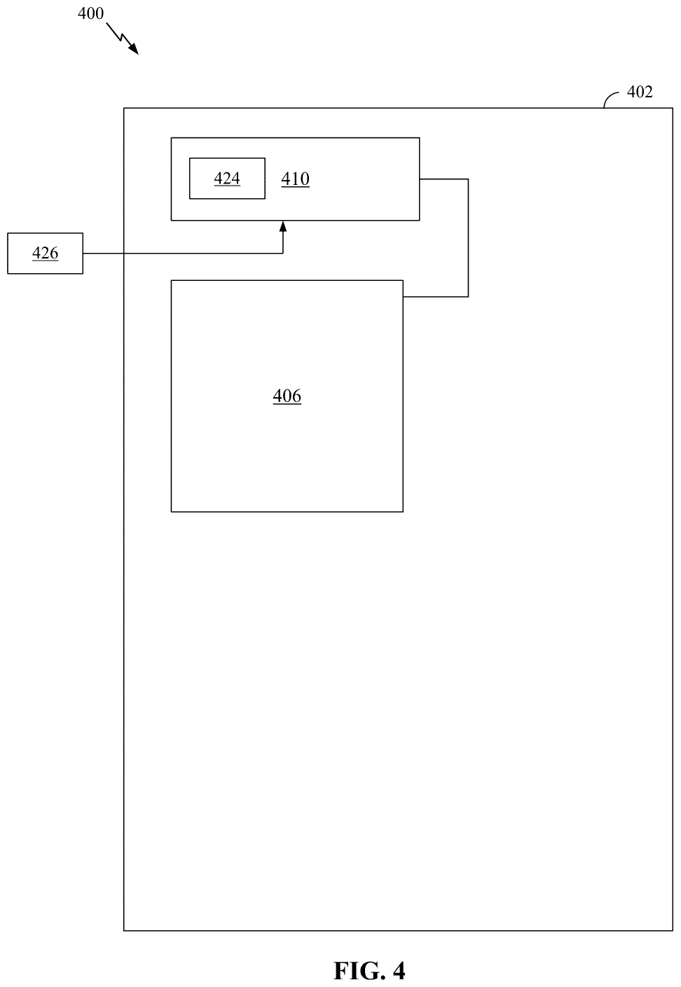

Referring now to FIG. 4, a block diagram of an example microbial control system 400 in accordance with one or more aspects of the present disclosure is shown. The system 400 includes an enclosure 402 with at least one electronic component 406 positioned (at least partially) within the interior of the enclosure 402. An example of the electronic component(s) 406 may include a controller of an Automated SmartWash Analytical Platform (ASAP)™, available from SmartWash Solutions, LLC of Salinas, California, and described in U.S. Patent Application Publication No. 2018/0093901 to Brennan et al., filed on Oct. 3, 2017 and entitled “System for Controlling Water Used for Industrial Food Processing.” An oxidant generator 410 is included with the system 400 to distribute an oxidizing agent within the interior of the enclosure 402. As shown, if the oxidant generator 410 uses electrical power for operation, the oxidant generator 410 may receive electrical power from one or more sources, such as at about 120 volts of alternating current (VAC) at 60 Hz, 110 VAC at 50 Hz, or about 24 volts of direct current (VDC). For example, with reference to FIG. 4, the oxidant generator 410 may receive electrical power from the electronic component(s) 406, and more specifically from a voltage regulator or other power supply circuit in the electronic component(s) 406.

The oxidant generator 410 may additionally or alternatively receive electrical power from a power source separate from the electronic component(s) 406, such as from an internal power source 424 and/or from an external power source 426. The internal power source 424 may be positioned within the enclosure 402 and/or may be included within the oxidant generator 410. The internal power source 424 may be portable, such as a battery. Further, the internal power source 424 may be rechargeable. The external power source 426 may be external to the enclosure 402. The external power source 426 may be portable or non-portable, and in some aspects, the external power source 426 may be used to charge the internal power source 424.

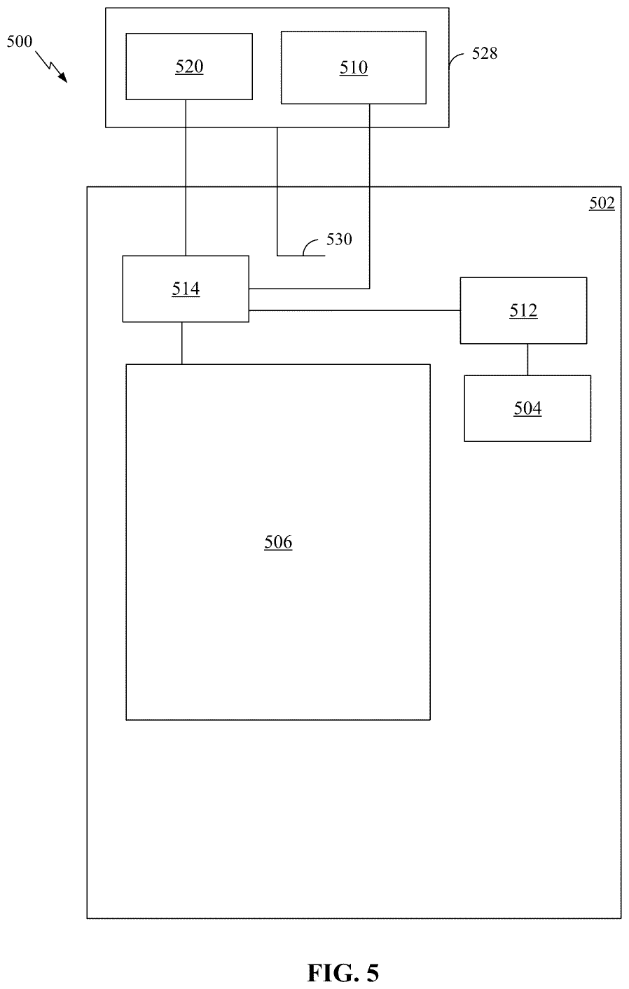

Referring now to FIG. 5, a block diagram of an example microbial control system 500 in accordance with one or more aspects of the present disclosure is shown. As with the above aspects, the system 500 includes an enclosure 502 with at least one electronic component 506 positioned (at least partially) within the interior of the enclosure 502. An example of the electronic component(s) 506 may include a controller of an Automated Smart Wash Analytical Platform (ASAP)™, available from SmartWash Solutions, LLC of Salinas, California, and described in U.S. Patent Application Publication No. 2018/0093901 to Brennan et al., filed on Oct. 3, 2017 and entitled “System for Controlling Water Used for Industrial Food Processing.” The system 500 also includes an oxidant generator 510 to generate an oxidizing agent and distribute the oxidizing agent within the interior of the enclosure 502. However, in this aspect, rather than having the oxidant generator 510 positioned within the enclosure 502, the oxidant generator 510 is positioned exterior to the enclosure 502 and is in fluid communication with the interior of the enclosure 502.

For example, the system 500 may further include an oxidant generator housing 528 with the oxidant generator 510 positioned within an interior of the oxidant generator housing 528. The interior of the oxidant generator housing 528 may be in fluid communication with the interior of the enclosure 502, such as through a flow line 530 (e.g., a tube or pipe), such that the oxidizing agent generated by the oxidant generator 510 is distributed to the interior of the enclosure 502 through the flow line 530. Further, a pressure source 520, such as a pump, may be used to provide a positive pressure to the interior of the oxidant generator housing 528, so as to facilitate fluid communication and pumping of the oxidizing agent from the oxidant generator housing 528 to the enclosure 502. As shown in FIG. 5, the pressure source 520 may be positioned within the interior of the oxidant generator housing 528 and provide the positive pressure to the interior of the enclosure 502 through the flow line 530.

The example microbial control system 500 may optionally include a controller 514 and a switch 512. As described above with reference to FIG. 3, the controller 514 may control operation of the oxidant generator 510 based on a signal from the switch 512. The switch 512 may generate a signal based on a position of an access cover 504, which may be closed to prevent access to the interior of the enclosure 502. The controller 514 may also control operation of the pressure source 520, based on the signal from the switch 512. The controller 514 may further receive other signals from a sensor within the enclosure 502 and/or a user interface and control the oxidant generator 510 and pressure source 520 based on those other signals.

In some aspects, the interior of the enclosure may include or be coupled to one or more other chemical or physical sources for microbial control. For example, other chemicals having antimicrobial properties, in addition or as an alternative to oxidizing agents such as ozone and/or chlorine dioxide discussed above, may be used. Further, a heat source may be included within or coupled to the enclosure for microbial control, such as by generating thermal energy to cause a temperature within the enclosure to be above a predetermined temperature (e.g., a threshold temperature), such that microorganisms cannot live within the enclosure.

One or more aspects of the present disclosure may be used to retrofit an existing enclosure including electronic component(s), such as to introduce microbial control for the enclosure. Some aspects of the present disclosure may include providing a kit or group of parts that may be used for microbial control for an existing enclosure. The kit may include a switch (e.g., switch 112 of FIG. 1) configured to operably couple to an access cover (e.g., access cover 104 of FIG. 1) of the enclosure such that the switch is in a first position when the access cover is in an open position with respect to an opening of the enclosure and the switch is in a second position when the access cover is in a closed position with respect to the opening, as described above. The kit may further include an oxidant generator, such as a UV light source, that is positionable within an interior of the enclosure. The oxidant generator of the kit may generate an oxidizing agent in a gaseous state for use within the interior of the enclosure and be operably coupled to the switch such that the switch causes the oxidant generator to operate when the switch is in a certain position (e.g., indicating the access cover is in the closed position). Further, the kit may include a controller (e.g., controller 314 of FIG. 3) and/or a pressure source (e.g., pressure source 320 of FIG. 3). The controller may be operably coupled to the oxidant generator and programmed to control the oxidant generator based upon the switch, a first signal from a timer, a second signal from a sensor, and/or a third signal from a user interface, for example. The pressure source may be configured to be placed in fluid communication with the interior of the enclosure to provide a positive pressure in the interior of the enclosure.

### Example Operations for Microbial Control

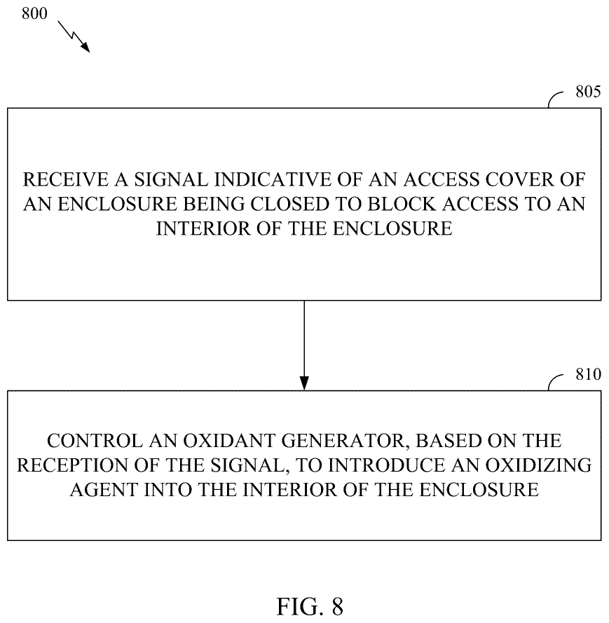

FIG. 8 is a flow diagram illustrating example operations 800 for controlling microbes, in accordance with certain aspects of the present disclosure. The operations 800 may be performed, for example, by a controller (e.g., controller 314, shown in FIG. 3) of a microbial control system, such as the microbial control system 300 shown in FIG. 3.

The operations 800 may begin, at block 805, with receiving a signal indicative of an access cover of an enclosure being closed to block access to an interior of the enclosure. For example, the controller 314 (see FIG. 3) may receive a signal from the switch 312 indicative of an access cover (e.g., a door or access cover 104 of FIG. 1) of the enclosure 302 being closed to block access to an interior of the enclosure 302.

At block 810, the operations 800 continue with controlling an oxidant generator, based on reception of the signal, to introduce an oxidizing agent into the interior of the enclosure. Continuing the example from above, the controller 314 (see FIG. 3) may control the oxidant generator 310, based on reception of a signal from the switch 312. The oxidant generator may be disposed in the enclosure or external to the enclosure.

Certain aspects of the present disclosure may be able to improve microbial control in enclosures, particularly for enclosures used within a microbial sensitive environment, such as within the food processing industry, the medical application industry, or the cosmetics industry. Certain aspects of the present disclosure may include electronic components positioned wholly or partially within the enclosure, but may also include or alternatively have other components commonly positioned within enclosures, such as mechanical components (e.g., valves or a manifold). Further, a microbial control system may be included with a motor control panel or enclosure, such as a variable drive motor control panel or enclosure, a logic controller panel or enclosure, a power distribution panel or enclosure, a process equipment control panel or enclosure, and/or a wash line or instrument control panel or enclosure (e.g., as described in U.S. Patent Application Publication No. 2018/0093901, entitled “System for Controlling Water Used for Industrial Food Processing,” filed on Oct. 3, 2017, and incorporated by reference herein in its entirety).

For example, an enclosure or a system capable of using an enclosure within the food processing industry may incorporate one or more aspects of the present disclosure. An enclosure may include one or more elements for controlling, testing, or detecting one or more substances used within a food processing system, such as controlling water chemistry (e.g., monitoring and controlling pH level and/or chlorine level for water used within a food processing system). These elements may include a sensor, a pump, a valve, a controller and/or a processor, and a human machine interface (HMI), such as a video display screen, to display information to a user. One or more of these elements may be positioned within the enclosure, and the enclosure may be portable, so as to be moved within a food processing plant, or may be non-portable and fixed in place (e.g., fixed to a larger structure). Certain aspects of the present disclosure may be incorporated within an enclosure used within a food processing system, such as by being retrofitted to be included within or operable with the enclosure. An oxidant generator, such as a UV light source, may be positioned within the interior of the enclosure to generate and distribute an oxidizing agent within the enclosure. A switch and a pump may be included and operable with the oxidant generator. Further, the oxidant generator may be electrically coupled to one or more pre-existing elements within the enclosure to receive electrical power. Thus, the present disclosure contemplates other elements and uses in addition or as alternatives to those provided and discussed above.

### Examples of Microbial Control in More Than One Enclosure

According to aspects of the present disclosure, because a microbial control device as described herein may allow certain portions of the microbial control device to be moved from one enclosure to another enclosure to assure microbial control without incurring the cost for a microbial control device for each enclosure, it may be desirable for the microbial control device as described herein to have additional functionalities that may not be cost-effective to include in non-portable microbial control devices (which may be installed in or on enclosures on a one-to-one basis).

For example, if an access port is included with each enclosure, the microbial control device can treat the interior of the enclosure, as previously described. If the microbial control device can be removed and the access port closed, then the microbial control device may be moved to another enclosure to provide additional microbial control. Access to power for the microbial control device may be provided through the same port or through an alternative connection. Access to power can also be provided externally, but would call for power being available near all enclosures to be treated.

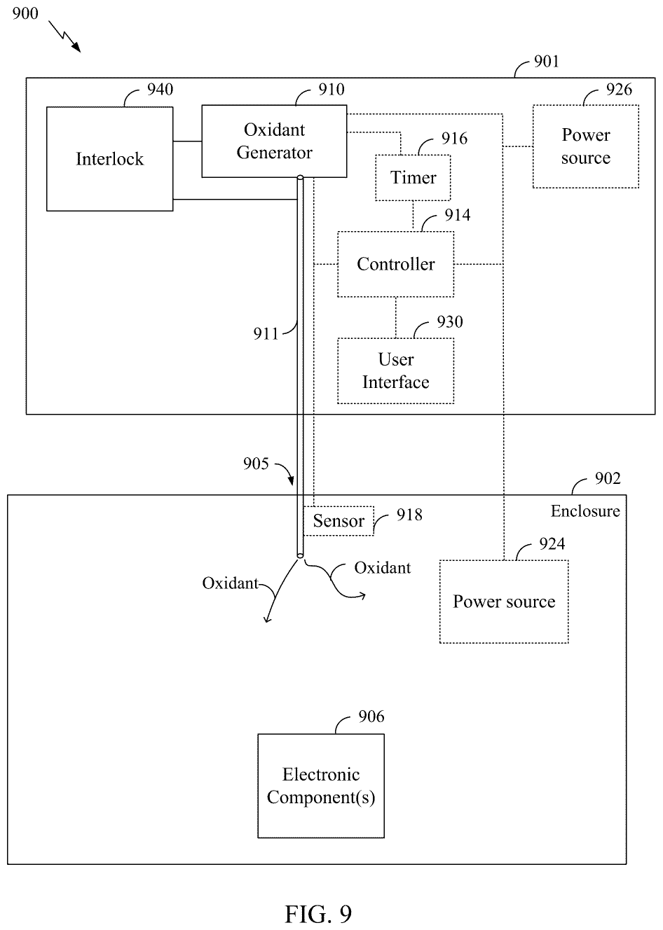

FIG. 9 is a block diagram of an example apparatus 900 for microbial control (e.g., a portable microbial control device) of an enclosure 902 in a processing facility, according to aspects of the present disclosure. The enclosure 902 may enclose one or more electronic components 906. The electronic component(s) 906 of FIG. 9 may be similar to the electronic component(s) 306 of FIG. 3.

The apparatus 900 may include a housing 901 for enclosing components disposed therein. The housing 901 may be composed of any of various suitable materials for use in a processing facility and may depend on the type of processing facility. Non-limiting example materials for the housing 901 may include stainless steel or plastic. The housing 901 may provide protection from the environment (e.g., from moisture and/or dust), a means to mount one or more of the components to avoid movement within the apparatus 900, and/or a means to carry and transport the apparatus 900.

The apparatus 900 includes an oxidant generator 910 configured to generate an oxidizing agent in a gaseous state, removably fluidly couple to an interior of the enclosure 902, and distribute the oxidizing agent in the gaseous state to the interior of the enclosure when the oxidant generator is fluidly coupled to the interior of the enclosure. For example, the oxidant generator 910 may include an ultraviolet (UV) light source. In this case, the UV light source may be disposed in a housing configured to push open an access port 905 of the enclosure 902, such that the UV light may be emitted into the enclosure. The access port 905 may be a separate entrance to the interior of the enclosure from entrance via an access cover (e.g., access cover 304) or door. Continuing this example, the UV light source may generate ozone from oxygen within the interior of the enclosure 902, and the generated ozone may be distributed within that enclosure. The housing (or at least an end of the housing) for the UV light source may have a dome, cylinder, cone, pyramid, or frustum shape, for example. In certain aspects, the housing for the UV light source (or at least a distal portion thereof) may be transparent or translucent. The housing for the UV light source may be composed of any of various suitable materials for UV light transmission therethrough, such as glass or plastic. In another example, the oxidant generator 910 is a gaseous oxidant generator, such as shown in and described with respect to FIGS. 7A-7C. In this example, the apparatus 900 may include a tube 911 (or other conduit) for fluid transfer. In certain aspects, the apparatus 900 may also include a plug (e.g., a quick connect plug) configured to removably connect to a receptacle (e.g., a quick connect receptacle) on the enclosure 902. In this example, when the quick connect plug is mated to the quick connect receptacle, the oxidant generator 910 may generate the gaseous oxidant, which passes through the tube 911 to the quick connect plug and receptacle and into the interior of the enclosure 902.

The apparatus 900 also includes an interlock 940 operable to prevent generation of the oxidizing agent by the oxidant generator 910 when the oxidant generator is not fluidly coupled to the interior of the enclosure 902 (e.g., via the housing for the UV light source or tube 911). For example, the interlock 940 may be implemented as a cover (e.g., an opaque cap) over the UV light source that only exposes the UV light source when the oxidant generator 910 is fluidly coupled to the interior of the enclosure 902. In another example, the interlock 940 may be implemented as a switch (e.g., a keyed plug switch) that is open unless the oxidant generator 910 is fluidly coupled to the interior of the enclosure 902. In this case, that switch may control power to or otherwise control activation of the oxidant generator 910. For example, the switch may be activated (e.g., closed) when the apparatus 900 is properly attached (e.g., electrically and mechanically coupled) to the enclosure 902, such that the oxidant generator is fluidly coupled to the interior of the enclosure. In yet another example, the interlock 940 may include at least one sensor (e.g., a photoelectric, capacitive, inductive, electromechanical, or magnetic sensor) that detects the presence of the enclosure 902 (e.g., a proximity sensor). This sensor may prevent the oxidant generator 910 from being activated when the enclosure 902 is not present or the oxidant generator is not fluidly coupled to the interior of the enclosure. In these and other similar manners, the interlock 940 may effectively check the connection of the apparatus 900 to the enclosure 902. The interlock 940 may turn on (and off) the oxidant generator 910 (or cause the oxidant generator to turn on (and off)) when the oxidant generator is (or is not) fluidly coupled to the interior of the enclosure 902.

In some aspects of the present disclosure, the oxidant generator 910 is configured to be at least partially positioned within the interior of the enclosure 902, such as when the oxidant generator is fluidly coupled to the interior of the enclosure. For example, at least a portion of the UV light source (and/or at least portion of the housing for the UV light source) may be inserted into or otherwise positioned inside the enclosure 902.

According to certain aspects of the present disclosure, the oxidant generator 910 comprises an ozone generator, and the oxidizing agent comprises ozone, such as shown in and described with respect to FIGS. 6A-6D. In some such aspects, the ozone generator comprises a UV light source, such as a mercury lamp or a UV light-emitting diode (LED). In some such aspects, the ozone generator includes an electrical discharge source having a pair of electrodes configured to generate an electric spark in a gap between the pair of electrodes (e.g., a spark gap).

In some aspects of the present disclosure, the oxidant generator 910 comprises a chlorine dioxide (ClO₂) generator, and the oxidizing agent comprises chlorine dioxide, as shown in and described with respect to FIGS. 7A-7C. In some such aspects, the chlorine dioxide generator includes a tablet (e.g., tablet 704 of FIG. 7B) configured to interact with an acid or water to generate the chlorine dioxide. In some aspects, the chlorine dioxide generator comprises a chlorite configured to interact with an acid to generate the chlorine dioxide (e.g., chlorite 710 and acid 720 of FIG. 7C). In this case, the chlorite may include at least one of sodium chlorite or potassium chlorite, and the acid may include at least one of hydrochloric acid or sulfuric acid.

According to certain aspects of the present disclosure, the apparatus 900 further includes a controller 914 operably coupled to the oxidant generator 910. The controller 914 of FIG. 9 may be similar to the controller 314 of FIG. 3. The controller 914 may be programmed to control the oxidant generator 910 based on at least one of a first signal from a timer 916, a second signal from a sensor 918, or a third signal from a user interface 930.

According to certain aspects of the present disclosure, the apparatus 900 further includes a sensor 918. The sensor 918 of FIG. 9 may be similar to the sensor 318 of FIG. 3. The sensor 918 may be configured to measure a presence or a concentration of the oxidizing agent within the interior of the enclosure 902. The sensor 918 may be operably coupled to the oxidant generator 910 (with or without an intervening component, such as the controller 914). In this manner, a signal generated by the sensor 918 is configured to at least one of: (i) prevent the oxidant generator 910 from operating when the concentration is above a first threshold or (ii) cause the oxidant generator 910 to operate when the concentration is below a second threshold.

In some aspects of the present disclosure, the apparatus 900 further includes a timer 916. The timer 916 of FIG. 9 may be similar to the timer 316 of FIG. 3. The timer 916 may be operably coupled to the oxidant generator 910 (with or without an intervening component, such as the controller 914). In this manner, the oxidant generator 910 may be configured to operate based on the timer 916.

In some aspects of the present disclosure, the oxidant generator 910 is configured to receive electrical power from a power source 924 of the enclosure 902. The power source 924 of FIG. 9 may be similar to the internal power source 424 of FIG. 4. In other aspects of the present disclosure, the oxidant generator 910 is configured to receive electrical power from a power source 926 separate from the enclosure 902. The power source 926 of FIG. 9 may be similar to the external power source 426 of FIG. 4. Although shown in FIG. 9 as being disposed inside the housing 901 of the apparatus 900, the power source 926 may be disposed outside the housing or may have components both inside and outside the housing. The power source 926 may be a portable power source (e.g., a battery or solar power) in some cases. In other cases, the power source 926 may plug into an AC electrical outlet (e.g., a wall outlet).

According to aspects of the present disclosure, a portable microbial control device may lock into a port of an enclosure in a manner to afford a firm connection such that the portable microbial control device prevents opening the enclosure until the portable microbial control device is removed. This removal may inactivate the portable microbial control device to protect user(s). The prevention of opening of the enclosure may be effected by having the housing of the portable microbial control device overlap an access cover of the enclosure or via various mechanical appendages to the portable microbial control device.

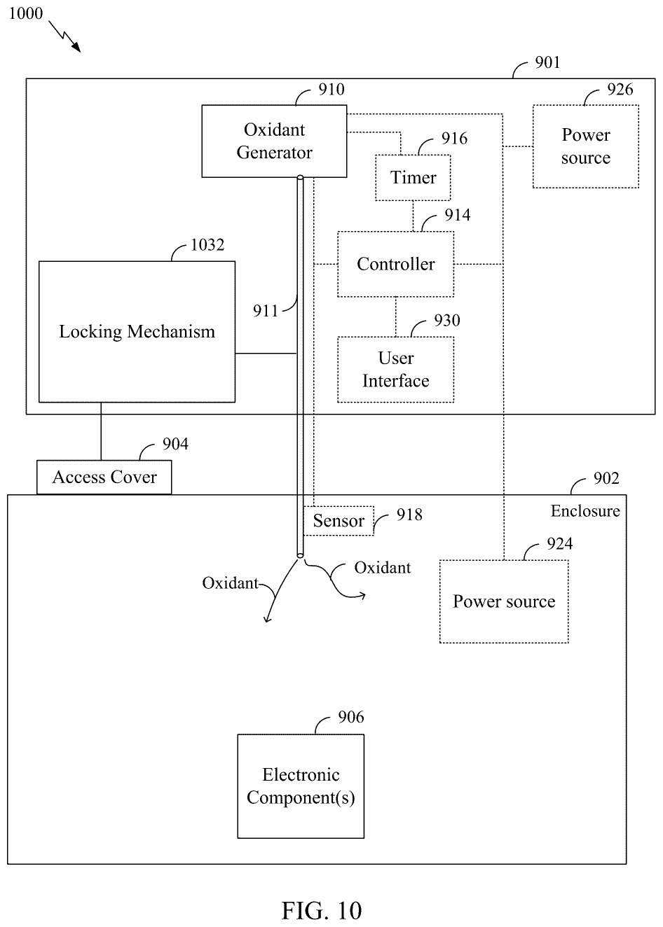

FIG. 10 is a block diagram of an example apparatus 1000 for microbial control (e.g., a portable microbial control device) of an interior of the enclosure 902 in a processing facility, where the enclosure 902 has an access cover 904 configured to be selectively opened to control access to the interior of the enclosure, according to aspects of the present disclosure. The apparatus 1000 of FIG. 10 is similar to the apparatus 900 of FIG. 9, and a description of common components with the same reference numerals is found above.

The apparatus 1000 of FIG. 10 also includes an interlock 1032 (also referred to herein as a “locking mechanism”) operable to prevent (e.g., physically prevent) the access cover 904 of the enclosure 902 from being opened when the oxidant generator 910 is fluidly coupled to the interior of the enclosure or when the apparatus 1000 is otherwise attached (e.g., mechanically and electrically coupled) to the enclosure. For example, the apparatus 1000 may attach to the enclosure 902 and block the access cover 904 from opening (or from being opened by a user) when a UV light source of the oxidant generator 910 is fluidly coupled to the interior of the enclosure, thereby functioning as the interlock 1032. In another example, the interlock 1032 may be implemented by at least one latch (e.g., a bolt or a key extending from the housing 901 of the apparatus 1000) or at least one magnet that effectively locks the access cover 904 of the enclosure 902 closed when the oxidant generator 910 is fluidly coupled to the interior of the enclosure.

The interlock 1032 may include or be implemented by at least one of: (i) the housing 901 of the apparatus 1000, wherein the housing blocks the access cover 904 from opening when the oxidant generator 910 is fluidly coupled to the interior of the enclosure 902; (ii) the housing 901 of the apparatus 1000, wherein the housing blocks access to a handle of the access cover 904 when the oxidant generator 910 is fluidly coupled to the interior of the enclosure 902; (iii) at least one latch that is configured to prevent the access cover 904 from opening when the oxidant generator 910 is fluidly coupled to the interior of the enclosure; or (iv) at least one magnet that is configured to hold the access cover 904 closed when the oxidant generator 910 is fluidly coupled to the interior of the enclosure 902. For some aspects, the interlock 1032 may include or be implemented by an attachment clamp. The clamp may be coupled to the housing 901 and may extend to the back of the enclosure 902 or to a fixed point on the enclosure such that the access cover 904 (e.g., a door) cannot be opened when the apparatus 1000 is properly coupled to the enclosure. For other aspects, the interlock 1032 may include or be implemented by an element that can be inserted through the access cover 904 and latches to a feature (e.g., a receptacle) in the interior of the enclosure 902. For other aspects, the interlock 1032 may actuate or otherwise operate (or prevent actuation/operation of) an existing closure mechanism (e.g., a handled latch or a keyed lock with a tap) in or on the access cover 904.

As described above, the timer 916 may be operably coupled to the oxidant generator 910 (with or without an intervening component, such as the controller 914). Additionally or alternatively, the timer 916 may be operably coupled to the interlock 1032 (with or without an intervening component, such as the controller 914). After the interlock 1032 is removed, the timer 916 (in conjunction with the controller 914, in some cases) can be used to determine and indicate when the access cover 904 may be opened, to prevent oxidant release outside of the enclosure 902.

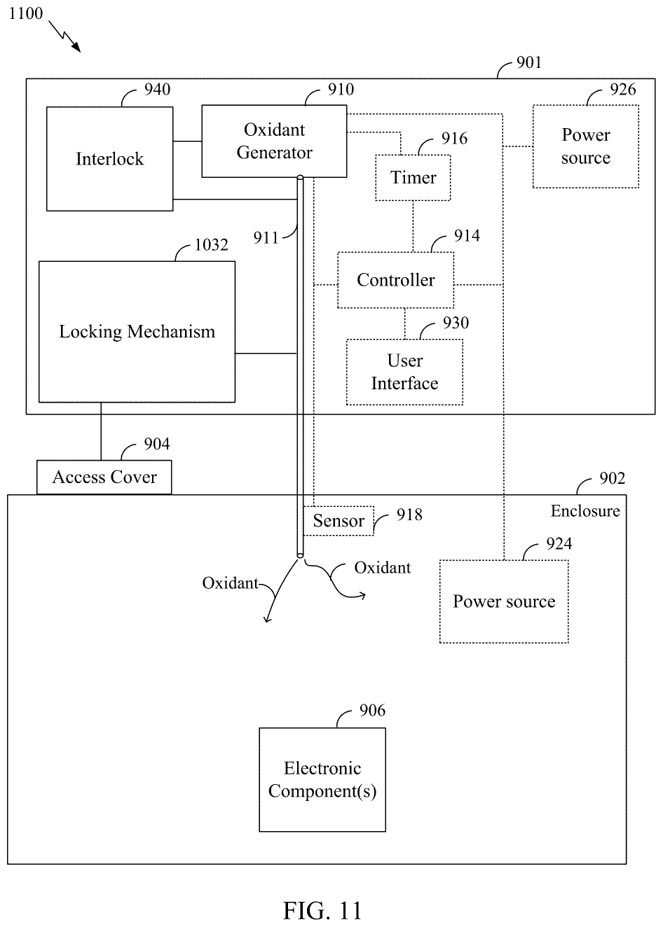

FIG. 11 shows a schematic illustration of an example apparatus 1100 for microbial control (e.g., a portable microbial control device) of an interior of the enclosure 902 in a processing facility, according to certain aspects of the present disclosure. The apparatus 1100 of FIG. 11 is similar to the apparatus 900 of FIG. 9 and the apparatus 1000 of FIG. 10, and a description of common components with the same reference numerals is found above.

The apparatus 1100 includes both the interlock 940 and the interlock 1032 (the locking mechanism). As described above, the interlock 940 may be operable to prevent generation of the oxidizing agent by the oxidant generator 910 when the oxidant generator is not fluidly coupled to the interior of the enclosure 902, and the interlock 1032 may be operable to prevent the access cover 904 of the enclosure from being opened when the oxidant generator is fluidly coupled to the interior of the enclosure. Thus, apparatus, such as apparatus 1100, with both the interlock 940 and the interlock 1032 may provide the highest safety level for users.

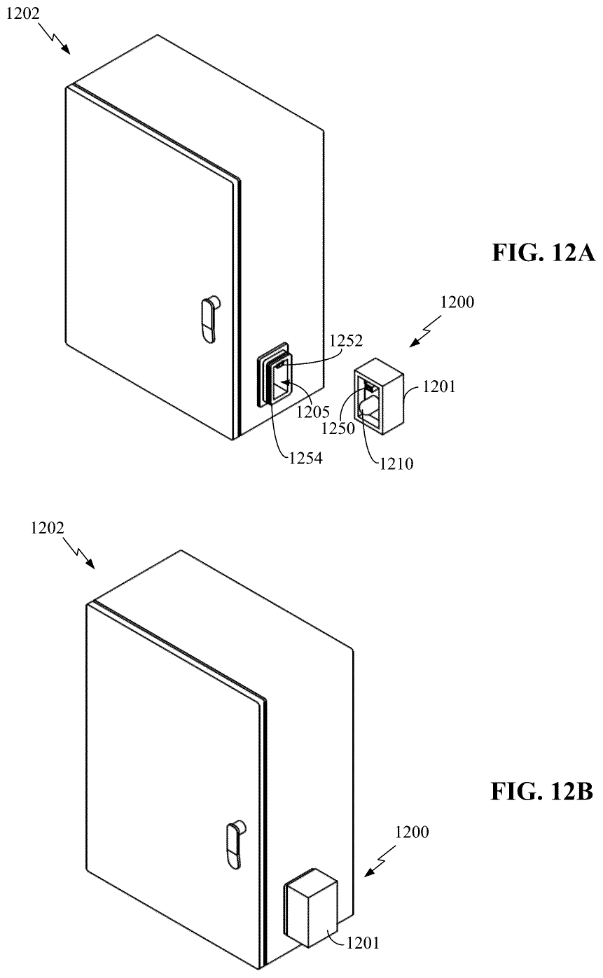

FIGS. 12A and 12B are perspective schematic views of an example portable microbial control device 1200 in different positions with respect to an enclosure 1202, in accordance with certain aspects of the present disclosure. In FIG. 12A, the portable microbial control device 1200 is illustrated being decoupled from a mount 1254 with an access port 1205 into the interior of the enclosure 1202. In FIG. 12B, the portable microbial control device 1200 is depicted as being coupled to the mount 1254, such that an oxidant generator 1210 (e.g., oxidant generator 910) enclosed by a housing 1201 of the device 1200 is removably fluidly coupled to the interior of the enclosure 1202 via the access port 1205.

The portable microbial control device 1200 of FIGS. 12A and 12B is similar to the apparatus 900 of FIG. 9, the apparatus 1000 of FIG. 10, and the apparatus 1100 of FIG. 11 and may contain any of the components described above, such as the controller 914, the interlock 940, and/or the interlock 1032. The housing 1201, access port 1205, and oxidant generator 1210 of FIGS. 12A and 12B may be similar to the housing 901, access port 905, and oxidant generator 910, respectively, of FIG. 9. As illustrated in FIG. 12A, the oxidant generator 1210 may be a UV lamp enclosed in a cylindrical housing with a dome-shaped end. The end of the housing for the UV lamp may extend from the housing 1201 for insertion into the access port 905 of the enclosure 1202. For certain aspects, the oxidant generator 1210 may have a cover (e.g., an opaque cap) or plug for preventing light or gas from escaping from the oxidant generator when not in use.

In addition, the portable microbial control device 1200 may have a power connector 1250. The power connector 1250 may be configured to mate with a power connector 1252 associated with the enclosure 1202, for supplying power to components in the portable microbial control device 1200 from a power source (e.g., internal power source 424 of FIG. 4) in the enclosure, when the oxidant generator 1210 is fluidly coupled to the interior of the enclosure. Although not shown, the portable microbial control device 1200 may also include a quick-connect plug for coupling to the enclosure 1202, as described above. To ensure proper connection (i.e., to prevent an improper connection), any of the plugs or receptacles described herein (e.g., the power connectors 1250, 1252 or the quick-connect plug) may be keyed (e.g., an asymmetrical tab or prong configuration for a plug with corresponding slits or cavities in the receptacle) such that the connectors may be mated in one and only one way.

### EXAMPLE CLAUSES

Implementation examples are described in the following numbered clauses:
Clause 1: An apparatus for microbial control in a processing facility, comprising: an oxidant generator configured to: generate an oxidizing agent in a gaseous state; removably fluidly couple to an interior of an enclosure of the processing facility; and distribute the oxidizing agent in the gaseous state to the interior of the enclosure when the oxidant generator is fluidly coupled to the interior of the enclosure; and an interlock configured to prevent generation of the oxidizing agent by the oxidant generator when the oxidant generator is not fluidly coupled to the interior of the enclosure.
Clause 2: The apparatus of Clause 1, wherein the oxidant generator is configured to be at least partially positioned within the interior of the enclosure when the oxidant generator is fluidly coupled to the interior of the enclosure.
Clause 3: The apparatus of Clause 1 or 2, wherein the oxidant generator comprises an ozone generator and wherein the oxidizing agent comprises ozone.
Clause 4: The apparatus of Clause 3, wherein the ozone generator comprises an ultraviolet (UV) light source.
Clause 5: The apparatus of Clause 4, wherein the UV light source comprises a mercury lamp or a light-emitting diode (LED).
Clause 6: The apparatus of Clause 3, wherein the ozone generator comprises an electrical discharge source having a pair of electrodes configured to generate an electric spark in a gap between the pair of electrodes.
Clause 7: The apparatus of Clause 1 or 2, wherein the oxidant generator comprises a chlorine dioxide generator and wherein the oxidizing agent comprises chlorine dioxide.
Clause 8: The apparatus of Clause 7, wherein the chlorine dioxide generator comprises a tablet configured to interact with an acid or water to generate the chlorine dioxide.
Clause 9: The apparatus of Clause 7 or 8, wherein the chlorine dioxide generator comprises a chlorite configured to interact with an acid to generate the chlorine dioxide.
Clause 10: The apparatus of Clause 9, wherein the chlorite comprises at least one of sodium chlorite or potassium chlorite and wherein the acid comprises at least one of hydrochloric acid or sulfuric acid.
Clause 11: The apparatus of any of Clauses 1 to 10, further comprising a sensor configured to measure a concentration of the oxidizing agent within the interior of the enclosure, wherein the sensor is operably coupled to the oxidant generator such that a signal generated by the sensor is configured to at least one of: prevent the oxidant generator from operating when the concentration is above a first threshold; or cause or allow the oxidant generator to operate when the concentration is below a second threshold.
Clause 12: The apparatus of any of Clauses 1 to 11, further comprising a timer operably coupled to the oxidant generator, wherein the oxidant generator is configured to operate based on a signal from the timer.
Clause 13: The apparatus of any of Clauses 1 to 12, further comprising a controller operably coupled to the oxidant generator and programmed to control the oxidant generator based on at least one of a first signal from a timer, a second signal from a sensor, or a third signal from a user interface.
Clause 14: The apparatus of any of Clauses 1 to 13, wherein the apparatus lacks an internal power source and wherein the oxidant generator is configured to receive electrical power from a power source of the enclosure.
Clause 15: The apparatus of any of Clauses 1 to 13, wherein the oxidant generator is configured to receive electrical power from a power source separate from the enclosure.
Clause 16: The apparatus of Clause 15, wherein the power source comprises a portable power source.
Clause 17: An apparatus for microbial control in a processing facility comprising an enclosure having an access cover configured to be selectively opened to control access to an interior of the enclosure, the apparatus comprising: an oxidant generator configured to: generate an oxidizing agent in a gaseous state; removably fluidly couple to the interior of the enclosure of the processing facility; and distribute the oxidizing agent in the gaseous state to the interior of the enclosure when the oxidant generator is fluidly coupled to the interior of the enclosure; and an interlock operable to prevent the access cover of the enclosure from being opened when the oxidant generator is fluidly coupled to the interior of the enclosure.
Clause 18: The apparatus of Clause 17, wherein the oxidant generator is configured to be at least partially positioned within the interior of the enclosure when the oxidant generator is fluidly coupled to the interior of the enclosure.
Clause 19: The apparatus of Clause 17 or 18, wherein the oxidant generator comprises an ozone generator and wherein the oxidizing agent comprises ozone.
Clause 20: The apparatus of Clause 19, wherein the ozone generator comprises an ultraviolet (UV) light source.
Clause 21: The apparatus of Clause 20, wherein the UV light source comprises a mercury lamp or a light-emitting diode (LED).
Clause 22: The apparatus of Clause 19, wherein the ozone generator comprises an electrical discharge source having a pair of electrodes configured to generate an electric spark in a gap between the pair of electrodes.
Clause 23: The apparatus of Clause 17 or 18, wherein the oxidant generator comprises a chlorine dioxide generator and wherein the oxidizing agent comprises chlorine dioxide.
Clause 24: The apparatus of Clause 23, wherein the chlorine dioxide generator comprises a tablet configured to interact with an acid or water to generate the chlorine dioxide.
Clause 25: The apparatus of Clause 23 or 24, wherein the chlorine dioxide generator comprises a chlorite configured to interact with an acid to generate the chlorine dioxide.
Clause 26: The apparatus of Clause 25, wherein the chlorite comprises at least one of sodium chlorite or potassium chlorite and wherein the acid comprises at least one of hydrochloric acid or sulfuric acid.
Clause 27: The apparatus of any of Clauses 17 to 26, further comprising a sensor configured to measure a concentration of the oxidizing agent within the interior of the enclosure, wherein the sensor is operably coupled to the oxidant generator such that a signal generated by the sensor is configured to at least one of: prevent the oxidant generator from operating when the concentration is above a first threshold; or cause or allow the oxidant generator to operate when the concentration is below a second threshold.
Clause 28: The apparatus of any of Clauses 17 to 27, further comprising a timer operably coupled to the oxidant generator, wherein the oxidant generator is configured to operate based on a signal from the timer.
Clause 29: The apparatus of any of Clauses 17 to 28, further comprising a controller operably coupled to the oxidant generator and programmed to control the oxidant generator based on at least one of a first signal from a timer, a second signal from a sensor, or a third signal from a user interface.
Clause 30: The apparatus of any of Clauses 17 to 29, wherein the apparatus lacks an internal power source and wherein the oxidant generator is configured to receive electrical power from a power source of the enclosure.
Clause 31: The apparatus of any of Clauses 17 to 29, wherein the oxidant generator is configured to receive electrical power from a power source separate from the enclosure.
Clause 32: The apparatus of Clause 31, wherein the power source comprises a portable power source.
Clause 33: The apparatus of any of Clauses 17 to 32, wherein the interlock comprises a housing of the apparatus, wherein the housing blocks the access cover from opening when the oxidant generator is fluidly coupled to the interior of the enclosure.
Clause 34: The apparatus of any of Clauses 17 to 32, wherein the interlock comprises a housing of the apparatus, wherein the housing blocks access to a handle of the access cover when the oxidant generator is fluidly coupled to the interior of the enclosure.
Clause 35: The apparatus of any of Clauses 17 to 32, wherein the interlock comprises a latch that is configured to prevent the access cover from opening when the oxidant generator is fluidly coupled to the interior of the enclosure.
Clause 36: The apparatus of any of Clauses 17 to 32, wherein the interlock comprises a magnet that is configured to hold the access cover closed when the oxidant generator is fluidly coupled to the interior of the enclosure.
Clause 37: The apparatus of any of Clauses 17 to 36, further comprising another interlock configured to prevent generation of the oxidizing agent by the oxidant generator when the oxidant generator is not fluidly coupled to the interior of the enclosure.
Clause 38: The apparatus of Clause 37, wherein the oxidant generator comprises an ultraviolet (UV) light source and wherein the other interlock comprises a cover over the UV light source.
Clause 39: The apparatus of Clause 37, wherein the other interlock comprises a switch.

### FURTHER CONSIDERATIONS

While the present disclosure has been described in detail in connection with a limited number of aspects, it should be readily understood that the present disclosure is not limited to such described aspects. Rather, the present disclosure can be modified to incorporate any number of variations, alterations, substitutions, combinations, sub-combinations, or equivalent arrangements not heretofore described, but which are commensurate with the scope of the present disclosure. Additionally, while various aspects of the present disclosure have been described, it is to be understood that aspects of the present disclosure may include only some of the described features.

The term “about” is intended to include the degree of error associated with measurement of the particular quantity based upon the equipment available at the time of filing the application. For example, “about” can include a range of ±8%, 5%, or 2% of a given value.

The terminology used herein is for the purpose of describing particular aspects only and is not intended to be limiting of the present disclosure. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, element components, and/or groups thereof.

While the present disclosure has been described with reference to exemplary aspects, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof.

Therefore, it is intended that the present disclosure not be limited to the particular aspect or aspects included as the best mode contemplated for carrying out the present disclosure, but that the present disclosure will include all aspects falling within the scope of the claims.

What is claimed is:

1. An apparatus for microbial control in a processing facility, comprising:
an oxidant generator configured to:
generate an oxidizing agent in a gaseous state;
removably fluidly couple to an interior of an enclosure of the processing facility; and
distribute the oxidizing agent in the gaseous state to the interior of the enclosure when the oxidant generator is fluidly coupled to the interior of the enclosure; and
an interlock configured to prevent generation of the oxidizing agent by the oxidant generator when the oxidant generator is not fluidly coupled to the interior of the enclosure.

2. The apparatus of claim 1, wherein the oxidant generator is configured to be at least partially positioned within the interior of the enclosure when the oxidant generator is fluidly coupled to the interior of the enclosure.

3. The apparatus of claim 1, wherein the oxidant generator comprises an ozone generator, wherein the oxidizing agent comprises ozone, wherein the ozone generator comprises an ultraviolet (UV) light source, and wherein the UV light source comprises a mercury lamp or a light-emitting diode (LED).

4. The apparatus of claim 1, wherein the oxidant generator comprises a chlorine dioxide generator, wherein the oxidizing agent comprises chlorine dioxide, and wherein the chlorine dioxide generator comprises a tablet configured to interact with an acid or water to generate the chlorine dioxide.

5. The apparatus of claim 1, further comprising a sensor configured to measure a concentration of the oxidizing agent within the interior of the enclosure, wherein the sensor is operably coupled to the oxidant generator such that a signal generated by the sensor is configured to at least one of:
prevent the oxidant generator from operating when the concentration is above a first threshold; or
cause or allow the oxidant generator to operate when the concentration is below a second threshold.

6. The apparatus of claim 1, further comprising a controller operably coupled to the oxidant generator and programmed to control the oxidant generator based on at least one of a first signal from a timer, a second signal from a sensor, or a third signal from a user interface.

7. The apparatus of claim 1, wherein the oxidant generator is configured to receive electrical power from a power source separate from the enclosure and wherein the power source comprises a portable power source.

8. An apparatus for microbial control in a processing facility comprising an enclosure having an access cover configured to be selectively opened to control access to an interior of the enclosure, the apparatus comprising:
an oxidant generator configured to:
generate an oxidizing agent in a gaseous state;
removably fluidly couple to the interior of the enclosure of the processing facility; and
distribute the oxidizing agent in the gaseous state to the interior of the enclosure when the oxidant generator is fluidly coupled to the interior of the enclosure; and
an interlock operable to prevent the access cover of the enclosure from being opened when the oxidant generator is fluidly coupled to the interior of the enclosure.

9. The apparatus of claim 8, wherein the oxidant generator is configured to be at least partially positioned within the interior of the enclosure when the oxidant generator is fluidly coupled to the interior of the enclosure.

10. The apparatus of claim 8, wherein the oxidant generator comprises an ozone generator, wherein the oxidizing agent comprises ozone, and wherein the ozone generator comprises an electrical discharge source having a pair of electrodes configured to generate an electric spark in a gap between the pair of electrodes.

11. The apparatus of claim 8, wherein the oxidant generator comprises a chlorine dioxide generator, wherein the oxidizing agent comprises chlorine dioxide, and wherein the chlorine dioxide generator comprises a tablet configured to interact with an acid or water to generate the chlorine dioxide.

12. The apparatus of claim 8, further comprising a sensor configured to measure a concentration of the oxidizing agent within the interior of the enclosure, wherein the sensor is operably coupled to the oxidant generator such that a signal generated by the sensor is configured to at least one of:
prevent the oxidant generator from operating when the concentration is above a first threshold; or
cause or allow the oxidant generator to operate when the concentration is below a second threshold.

13. The apparatus of claim 8, further comprising a timer operably coupled to the oxidant generator, wherein the oxidant generator is configured to operate based on a signal from the timer.

14. The apparatus of claim 8, further comprising a controller operably coupled to the oxidant generator and programmed to control the oxidant generator based on at least one of a first signal from a timer, a second signal from a sensor, or a third signal from a user interface.

15. The apparatus of claim 8, wherein the apparatus lacks an internal power source and wherein the oxidant generator is configured to receive electrical power from a power source of the enclosure.

16. The apparatus of claim 8, wherein the interlock comprises a housing of the apparatus and wherein the housing blocks at least one of:
the access cover from opening when the oxidant generator is fluidly coupled to the interior of the enclosure; or
access to a handle of the access cover when the oxidant generator is fluidly coupled to the interior of the enclosure.

17. The apparatus of claim 8, wherein the interlock comprises a latch that is configured to prevent the access cover from opening when the oxidant generator is fluidly coupled to the interior of the enclosure.

18. The apparatus of claim 8, wherein the interlock comprises a magnet that is configured to hold the access cover closed when the oxidant generator is fluidly coupled to the interior of the enclosure.

19. The apparatus of claim 8, further comprising another interlock configured to prevent generation of the oxidizing agent by the oxidant generator when the oxidant generator is not fluidly coupled to the interior of the enclosure.

20. The apparatus of claim 19, wherein the oxidant generator comprises an ultraviolet (UV) light source and wherein the other interlock comprises a cover over the UV light source.
